(19) United States
(12) Patent Application Publication
(10) Pub. No.: US 2025/0000974 A1
(43) Pub. Date: Jan. 2, 2025

(54) COMBINATION OF HUMAN NATURAL KILLER CELLS AND MACROPHAGES FOR CANCER THERAPY

(71) Applicant: The Regents of the University of California, Oakland, CA (US)

(72) Inventors: Benjamin Goldenson, San Diego, CA (US); Dan S. Kaufman, La Jolla, CA (US)

(21) Appl. No.: 18/703,963

(22) PCT Filed: Oct. 26, 2022
(86) PCT No.: PCT/US2022/047840

§ 371 Date: Apr. 23, 2024

Related U.S. Application Data
(60) Provisional application No. 63/272,305, filed on Oct. 27, 2021.

(51) Int. Cl. A61K 39/00; C07K 16/28

(52) U.S. Cl. CPC A61K 39/4613; A61K 39/001111; A61K 39/4614; C07K 16/2803; C07K 2317/24; C07K 2317/732

(57) ABSTRACT

Compositions and combination cellular therapies with natural killer (NK) cells and macrophages for treating disease such a leukemia are disclosed herein. The compositions and therapeutic methods of administration may optionally include antibodies, such as anti-CD47 or other SIRP signaling inhibitors. The NK and macrophage cells may be produced from stem cells, and may optionally be produced from induced pluripotent stem cells (iPSCs), where the NK and macrophage cells may have enhanced antibody-dependent cellular cytotoxicity (ADCC). These immune cells may also be incorporated into pharmaceutical compositions. Methods for making the immune cells and pharmaceutical compositions, and methods of use thereof, are also provided.

### CROSS REFERENCE TO RELATED APPLICATIONS

This application claims priority to U.S. Provisional Application No. 63/272,305 filed on Oct. 27, 2021, the entire contents of which are incorporated by reference.

### GOVERNMENT SPONSORSHIP

This invention was made with government support under grant No. U01CA217885 awarded by the National Institutes of Health. The government has certain rights in the invention.

### TECHNICAL FIELD

The present invention relates generally to the field of cellular therapy to treat disease.

### BACKGROUND

Adoptive cell therapy is rapidly gaining interest as a promising new method to treat cancer. In particular, chimeric antigen receptor (CAR) T cells, the only U.S. F.D.A.-approved lymphocyte-based adoptive cancer cell therapy to treat cancer recently approved in 2017, have shown remarkable efficacy in treating refractory B cell malignancies. Success of CAR-T cell therapy has fueled optimism for the development of more effective adoptive cell therapy options. Currently approved CAR-T treatment regimens rely on autologous transplantation of ex vivo modified and expanded T cells harvested through leukapheresis from the original patients. This process takes 3-4 weeks, and donor variability on the quality of harvested T cells from each individual patient can widely affect treatment outcome. Furthermore, some patients receiving CAR-T cell therapy experience potentially lethal side effects, notably cytokine release syndrome (CRS) and neurotoxicity. Thus, development of standardized, “off-the-shelf” cell therapy products with defined, consistent quality that can be administered into patients in a timely manner with minimal side effects is highly desirable and is of great commercial interest.

In this context, focus is turning to natural killer (NK) cells as a suitable cell source for “off-the-shelf” cell therapy. Unlike T cells, NK cells possess a native ability to kill tumors and virally infected cells without prior antigen priming. Furthermore, NK cells can be administered to patients across HLA allotypes, unlike T cells which require HLA matching to avoid graft-versus-host disease. Many trials utilizing adoptive transfer of allogeneic NK cells demonstrated complete remissions in patients with acute myelogenous leukemia (AML) who are refractory to standard chemotherapy. Another recent clinical study demonstrated effective treatment of lymphoid malignancies using allogeneic CAR-expressing NK cells, with minimal side effects. Thus, NK cells possess a number of advantages over T cells that enables them to be used as safe, effective, “off-the-shelf” adoptive cell therapy product to treat diverse malignancies.

As such, NK cells can be useful in adoptive cell therapies, however their use is often limited by biological constraints and results in suboptimal efficacy. Therefore, there is an unmet need for improved compositions comprising said cells and methods of their use.

### SUMMARY OF THE INVENTION

Disclosed herein are compositions and methods of prevention and treatment for a subject in need comprising administering to the subject an effective amount of natural killer (NK) cells and macrophages, both with and without inclusion of monoclonal antibodies targeting the CD47-SIRP pathway or other immune regulating pathways (such as PDT, PDL1 or CTLA4), which leads to the death of cancer cells, including acute myeloid leukemia (AML) and multiple myeloma cells.

In embodiments, the disclosure provides a method of treating or preventing a disease or disorder in a subject in need thereof, comprising administering to the subject an effective amount of natural killer (NK) cells and macrophages.

In embodiments, the method further comprising administering to the subject an effective amount of an antibody blocking an immune regulatory pathway. In embodiments, the antibody is anti-CD47, anti-PD1, anti-PDL1 or anti-CTLA4.

In embodiments, the subject suffers from a cancer, such as AML and multiple myeloma.

In embodiments, the NK cells and the macrophages are derived from induced pluripotent stem cell (iPSC)-derived immune cells, peripheral blood (PB)-derived immune cells or cord blood (CB)-derived immune cells.

In embodiments, the invention provides a purified cell composition comprising at least partially isolated natural killer (NK) cells and macrophages. In embodiments, the purified cell composition further comprises an antibody blocking an immune regulatory pathway, including but not limited to an anti-CD47, anti-PD1, anti-PDL1 or anti-CTLA4 antibody.

### BRIEF DESCRIPTION OF THE DRAWINGS

FIG. 1 depicts the system of differentiation of NK cells and macrophages from iPSCs.

FIG. 2 is a graph comparing of control and antibody stained samples from standard iPSC-NK cells and iPSC-macrophages phenotypes.

FIG. 3A depicts a proposed mechanism for a combination therapy of iPSC-NK cells and iPSC-macrophages.

FIG. 3B depicts a proposed mechanism for a combination therapy of iPSC-NK cells, iPSC-macrophages, and anti-CD47 antibodies.

FIG. 4A is a graph depicting the improved efficacy of adding iPSC-macrophages and/or anti-CD47 antibodies to iPSC-NDK cells for killing AML cells.

FIG. 4B is a graph depicting the improved efficacy of adding iPSC-macrophages and/or anti-CD47 antibodies to iPSC-NDK cells for killing AML cells.

FIG. 4C is a graph depicting the improved efficacy of adding iPSC-macrophages and/or anti-CD47 antibodies to iPSC-NDK cells for killing multiple myeloma cells.

FIG. 5A is a graph comparing the efficacy of iPSC-NK cells, iPSC-NK cells+iPSC-macrophages, and iPSC-NK cells+iPSC-macrophages+anti-CD47 antibody, and iPSC-NK cells+iPSC-macrophages+anti-CD47 antibody+anti-SIRPα antibody in killing AML cells.

FIG. 5B is a graph depicting that increased anti-tumor activity of combined iPSC-NK cells+iPSC-macrophages+anti-CD47 therapy is mediated by the anti-SIRPα antibody.

FIG. 6A is a graph comparing the efficacy of iPSC-NK cells, iPSC-NK cells+anti-CD47 antibody+FcX, and iPSC-NK cells+anti-CD47 antibody in killing AML cells.

FIG. 6B is a graph depicting that the anti-tumor effect of the anti-CD47 antibody is not mediated through ADCC.

FIG. 7A is a graph comparing the efficacy of iPSC-NK cells, contacted iPSC-NK cells+iPSC-macrophages, and separated iPSC-NK cells+iPSC-macrophages.

FIG. 7B is a graph depicting that the anti-tumor effect of the iPSC-NK cells and iPSC-macrophages requires physical contact between the iPSC-NK cells and iPSC-macrophages.

### DETAILED DESCRIPTION

The present disclosure provides a cellular therapy treatment for cancer and other diseases comprising administration to a subject in need thereof a combination of natural killer (NK) cells and macrophages, with or without the addition of monoclonal antibodies targeting the CD47-SIRP pathway or other immune regulatory pathways. This invention can be applied to any NK cell population, including (but not limited to) those induced, derived or isolated from human embryonic stem cells, human peripheral blood or umbilical cord blood.

In embodiments, the invention provides an iPSC-derived NK cell and macrophage cellular therapy, and optional monoclonal antibody combination treatment, approach to produce a targeted off the-shelf immunotherapy for cancer.

The combination treatment of IPSC-derived NK cells and macrophages and anti-CD47 antibodies can be utilized for improved cancer therapies. For example, this combination treatment can be used against AML and multiple myeloma cells with demonstrated efficacy. There are now many ongoing clinical trials of NK cells (including iPSC-derived NK cells) against diverse tumor types. Macrophage-based therapies are also now entering clinical trials. Having both of these cell populations derived from a standardized iPSC source administered together provides an unexpected advantage compared to use of these cells isolated from peripheral blood (PB) or cord blood (CB). Additionally, agents that block immune regulatory pathways, such as anti-CD47 antibody, or otherwise stimulate the immune cells, such as (but not limited to) anti-PD1, anti-PDL1 or anti-CTLA4 antibodies, can also mediate improved anti-tumor activity with the combined NK cell and macrophage approach.

Various further aspects and embodiments of the disclosure are provided by the following description.

Unless defined otherwise, all technical and scientific terms used herein have the same meanings as commonly understood by one of ordinary skill in the art to which the present disclosure belongs. Any materials and methods similar or equivalent to those described herein can be used to practice the present invention. The practice of the present invention may employ conventional techniques of molecular biology (including recombinant techniques), microbiology, cell biology, biochemistry and immunology, which are within the skill of the art. Such techniques are explained fully in the literature, such as Molecular Cloning: A Laboratory Manual, second edition (Sambrook et al, 1989) Cold Spring Harbor Press; Oligonucleotide Synthesis (M J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C C. Blackwell, eds.); Gene Transfer Vectors for Mammalian Cells (J. M. Miller and M. P. Calos, eds., 1987); Current Protocols in Molecular Biology (F. M. Ausubel et al, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al, eds., J. B. Lippincott Company, 1993). All publications, patents, and patent applications mentioned in this specification are herein incorporated by reference to the same extent as if each individual publication, patent, or patent application was specifically and individually indicated to be incorporated by reference.

Although any methods and materials similar or equivalent to those described herein can be used in the practice of the present invention, the exemplary methods, devices, and materials are described herein.

For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, an engineered immune cell, a pharmaceutical composition, and/or a method that “comprises” a list of elements (e.g., components, features, or steps) is not necessarily limited to only those elements (or components or steps), but may include other elements (or components or steps) not expressly listed or inherent to the engineered immune cell, pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It should be understood that the terms “a” and “an” as used herein refer to “one or more” of the enumerated components unless otherwise indicated. The use of the alternative (e.g., “or”) should be understood to mean either one, both, or any combination thereof of the alternatives.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment.

It will be further understood that there are a number of values disclosed therein, and that each value is also herein disclosed as “about” that particular value in addition to the value itself. In embodiments, “about” can be used to mean, for example, a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In various embodiments, the term “about” or “approximately” refers a range of quantity, level, value, number, frequency, percentage, dimension, size, amount, weight or length ±15%, ±10%, ±9%, ±8%, ±7%, ±6%, ±5%, 4%, ±3%, ±2%, or ±1% about a reference quantity, level, value, number, frequency, percentage, dimension, size, amount, weight or length.

As used herein, “induced pluripotent stem cell” or “iPSC cell” or “iPSCs” are used to refer to cells, derived from somatic cells, that have been reprogrammed back to an pluripotent state that are capable of proliferation, selectable differentiation, and maturation.

As used herein, “peripheral blood” or “peripheral blood cell” is used to refer to cells that originate from circulating blood and comprise hematopoietic stem cells that are capable of proliferation, selectable differentiation, and maturation.

As used herein, “cord blood cell” is used to refer to cells that originate from the umbilical cord and placenta and comprise hematopoietic stem cells that are capable of proliferation, selectable differentiation, and maturation.

As used herein, and unless otherwise specified, a “natural killer cell” or “NK cell” is used to refer to cells that are cytotoxic lymphocytes that constitute a major component of the innate immune system. In humans a natural killer cell usually expresses the surface markers CD16 (FCyRIII) and CD56. NK cells are cytotoxic; small granules in cytoplasm that contain special proteins such as perform and proteases known as granzymes. NK cells provide rapid responses to virally infected cells and respond to transformed cells. Upon release in close proximity to a cell slated for killing, perforin forms pores in the cell membrane of the target cell through which the granzymes and associated molecules can enter, inducing apoptosis. Thus, NK cells may act as effectors of lymphocyte population in anti-tumor and anti-infection immunity.

Typically, immune cells detect peptides from pathogens presented by Major Histocompatibility Complex (MHC) molecules on the surface of infected cells, triggering cytokine release, causing lysis or apoptosis. NK cells are unique, however, as they have the ability to recognize stressed cells regardless of whether peptides from pathogens are present on MHC molecules. They were named “natural killers” because of the initial notion that they do not require prior activation in order to kill a target. NK cells are large granular lymphocytes (LGL) and are known to differentiate and mature in the bone marrow from where they then enter into the circulation. In some embodiments, the NK cells are characterized by being CD56+ CD3−. In some embodiments, the NK cells are characterized by being CD56+ CD45+. In some embodiments, the NK cells are characterized by being CD56+ CD45+ CD3−. In some embodiments, the NK cells are characterized by being CD56+ CD45+ CD33−. In some embodiments, NK cells are characterized by being CD56+CD45+ CD3− CD33−. In some embodiments, NK cells are characterized by being CD56+CD94+ NKG2D+ NKp44+ NKp46+. In some embodiments, NK cells are characterized by being CD56+ NKG2D+ NKp44+ NKp46+. In some embodiments, NK cells are characterized by being NKp30+ NKp44+ NKp46+. In some embodiments, NK cells are characterized by being NKp30+. In some embodiments, NK cells are characterized by being NKp44+. In some embodiments, NK cells are characterized by being NKp46+. In some embodiments, NK cells are characterized by being CD94+ NKG2+. In some embodiments, NK cells are characterized by being inhibitory killer-immunoglobulin-like receptor (KIR+).

As used herein, and unless otherwise specified, a “macrophage” is used to refer to cells that are involved in the detection, phagocytosis and destruction of bacteria and other harmful organisms. In addition, they can also present antigens to T cells and initiate inflammation by releasing cytokines that activate other cells. Macrophages typically express phenotypic antigens that include CD11b, CD14, CD68, CD86, SIRPα, and HLA class II antigens. Important roles of these cell surface molecules include mediating cell signaling, phagocytosis, and functioning as toll-like-receptors, lectin receptors, and scavenger receptors. Macrophages can also mediate antibody dependent cell cytotoxicity (ADCC) and/or antibody dependent cell phagocytosis (ADCP) via expression of Fc receptors CD16, CD32, and/or CD64.

In some embodiments, the immune cell is a human immune cell.

In an aspect, the disclosure provides a purified cell composition comprising one or more of the NK and macrophage immune cells.

As used herein, a composition containing a “purified cell population” or “purified cell composition” means that at least 30%, 50%, 60%, typically at least 70%, and more preferably 80%, 90%, 95%, 98%, 99%, or more of the cells in the composition are of the identified type.

In some embodiments, the cells described herein are further engineered immune cells. In some embodiments, the engineered immune cell is a natural killer (NK) cell or a macrophage. As used herein, “engineered” or “genetically modified” or “transformed” are used interchangeably, wherein a cell has been manipulated by means of molecular reprogramming of a genomic sequence (e.g. by insertion, deletion, or substitution). Said cells include the primary transformed cell and progeny derived therefrom without regard to the number of passages. Progeny may not be completely identical in nucleic acid content to a parent cell and may contain mutations. Mutant progeny that have the same function or biological activity as screened or selected for in the originally transformed cell are included herein.

The engineered immune cells may exhibit upregulation and/or stabilization of certain cell surface markers compared to wildtype cell counterparts. In some embodiments, the engineered immune cells exhibit stabilization of CD16 compared to wildtype cell counterparts. In some embodiments, the engineered immune cells exhibit stabilization of CD62L compared to wild type counterparts. In some embodiments, the engineered immune cells exhibit enhanced surface expression of TNFα compared to wild type counterparts.

Genome editing tools may be used to engineer and/or manipulate cells. In some embodiments, the immune cell of the disclosure may be engineered with either CRISPR, TALEN, or ZFN genome editing tools.

Genome editing tools such as the clustered regularly interspaced short palindromic repeats (CRISPR) system may be used to genetically modify cells. CRISPR can be used in a wide variety of organisms (e.g., used to add, disrupt, or change the sequence of specific genes). The targeting sequence can be designed or chosen using computer programs known to persons of ordinary skill in the art. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like.

The immune cells described herein can be modified using methods known in the art. The various gene editing systems described herein may be used to modify the immune cell to delete, inactivate, reduce expression, or otherwise inhibit function of a target gene or a target gene product.

The term “nucleic acid” or “polynucleotide”, includes DNA and RNA such as genomic DNA, cDNA and mRNA, or combinations thereof. The nucleic acid may comprise, in addition to the sequence enabling the genetic modifications of the disclosure, further sequences such as those required for the transcription and/or translation of the nucleic acid enabling said genetic modifications. This may include a promoter, enhancer, transcription and/or translation initiation and/or termination sequences, selection markers, sequences protecting or directing the RNA and/or enabling the genetic modifications within the cell. The selection and combination of these sequences is within the knowledge of the person skilled in the art and may be selected in accordance with the cell the nucleic acid is intended for.

Generally, techniques for differentiating an induced pluripotent cell involve modulation of specific cellular pathways, either directly or indirectly, using polynucleotide-, polypeptide- and/or small molecule-based approaches. The developmental potency of a cell may be modulated, for example, by contacting a cell with one or more modulators. “Contacting”, as used herein, can involve culturing cells in the presence of one or more factors (such as, for example, small molecules, proteins, peptides, etc.). In some embodiments, a cell is contacted with one or more agents to induce cell differentiation. Such contact, may occur for example, by introducing the one or more agents to the cell during in vitro culture. Thus, contact may occur by introducing the one or more agents to the cell in a nutrient cell culture medium. The cell may be maintained in the culture medium comprising one or more agents for a period sufficient for the cell to achieve the differentiation phenotype that is desired.

Differentiation of stem cells requires a change in the culture system, such as changing the stimuli agents in the culture medium or the physical state of the cells. A conventional strategy utilizes the formation of embryoid bodies (EBs) as a common and critical intermediate to initiate the lineage-specific differentiation. EBs are three-dimensional clusters that have been shown to mimic embryo development as they give rise to numerous lineages within their three-dimensional area. Through the differentiation process simple EBs (for example, aggregated pluripotent stem cells elicited to differentiate) continue maturation and develop into a cystic EB at which time, they are further processed to continue differentiation. EB formation is initiated by bringing pluripotent stem cells into close proximity with one another in three-dimensional multilayered clusters of cells. To promote EB development, the pluripotent stem cell aggregates require further differentiation cues, as aggregates maintained in pluripotent culture maintenance medium do not form proper EBs. This may be followed by additional stimulation differentiating the iPSCs to hematopoietic cells and then to convert the hematopoietic progenitor cells into natural killer (NK).

As used herein, “differentiate” or “differentiated” are used to refer to the process and conditions by which immature (unspecialized) cells acquire characteristics becoming mature (specialized) cells thereby acquiring particular form and function. Stem cells (unspecialized) are often exposed to varying conditions (e.g., growth factors and morphogenic factors) to induce specified lineage commitment, or differentiation, of said stem cells. The process by which an unspecialized (“uncommitted”) or less specialized cell acquires the features of a specialized cell such as, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

“Culture” or “cell culture” refers to the maintenance, growth and/or differentiation of cells in an in vitro environment. “Cell culture media,” “culture media” (singular “medium” in each case), “supplement” and “media supplement” refer to nutritive compositions that cultivate cell cultures.

“Cultivate,” or “maintain,” refers to the sustaining, propagating (growing) and/or differentiating of cells outside of tissue or the body, for example in a sterile plastic (or coated plastic) cell culture dish or flask. “Cultivation,” or “maintaining,” may utilize a culture medium as a source of nutrients, hormones and/or other factors helpful to propagate and/or sustain the cells.

Multipotent hematopoietic stem cells provide the basis of two major progenitor cell lineages. The first cell lineage is the common lymphoid progenitor cell lineage, wherein a multipotent hematopoietic stem cell (hemocytoblast) differentiates into a lymphoid progenitor cell, which has the capability to further differentiate into a natural killer cell, T lymphocyte, or B lymphocyte; or differentiate even further from a B lymphocyte to a plasma cell. The other major cell lineage is the common myeloid progenitor cell lineage, wherein a hemocytoblast differentiates into a myeloid progenitor cell, which has the capability to further differentiate into a megakaryocyte, erythrocyte, platelet, mast cell, or myeloblast; or differentiate even further from a myeloblast to a basophil, neutrophil, eosinophil, or monocyte; or yet further differentiate from a monocyte to a macrophage.

As used herein, the term “pluripotent” refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. As such, the term “pluripotent stem cell”, as used herein, refers to a subset of undifferentiated cells that are capable of giving rise to hematopoietic stem and progenitor cells via hematopoietic transition.

In an aspect, the disclosure provides a pharmaceutical composition comprising the engineered immune cell of the disclosure and one or more pharmaceutically acceptable excipients or diluents.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable diluent or excipient” or “pharmaceutically acceptable carrier” refers to an excipient, diluent, preservative, solubilizer, emulsifier, adjuvant, and/or vehicle with which an NK cell of the disclosure, is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable diluent or excipient” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

Formulations of a pharmaceutical composition suitable for administration typically generally comprise the active ingredient combined with a pharmaceutically acceptable diluents or excipients, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. Formulations may also include aqueous solutions which may contain excipients such as salts, carbohydrates and buffering agents or sterile, pyrogen-free, water. Exemplary administration forms may include solutions or suspensions in sterile aqueous solutions, for example, aqueous propylene glycol or dextrose solutions. Such dosage forms can be suitably buffered, if desired.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, and/or aromatic substances and the like which do not deleteriously interact with the formulation. In some embodiments, the pharmaceutical composition comprises said NK cells and macrophages in combination with other therapeutically active agents. In some embodiments, the pharmaceutical composition comprises said NK cells and macrophages in combination with antibodies specific to a disease cell phenotype. In some embodiments, the disease cell phenotype is that of a malignant cell. In some embodiments, the disease cell phenotype is that of a viral infection.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

In an aspect, the disclosure provides a kit comprising the immune cell of the disclosure, with or without antibodies of the disclosure, or the pharmaceutical composition of the disclosure and instructions for use.

The present invention provides NK and macrophage immune cells derived from a renewable source of iPSCs. These cells provide a promising use for therapies in conjunction with therapeutic antibodies to effectively treat refractory malignancies and potentially other diseases, such as ALM and MM.

In an aspect, the disclosure provides a method of treating or preventing a disease or disorder in a subject in need thereof, comprising administering the immune cells of the disclosure or the pharmaceutical composition of the disclosure to the subject. In some embodiments, the disease or disorder is a malignancy. In some embodiments, the malignancy comprises a tumor-associated antigen.

The terms “subject,” “patient” and “individual” are used interchangeably herein to refer to a vertebrate, preferably a mammal, more preferably a human. Tissues, cells, and their progeny of a biological entity obtained in vivo or cultured in vitro are also encompassed. A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

In some embodiments, administering comprises administering a therapeutically effective amount to a subject.

As used herein, the term “amount” refers to “an amount effective” or “an effective amount” of a cell to achieve a beneficial or desired prophylactic or therapeutic result, including clinical results. As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent. In some embodiments, the immune cells or pharmaceutical composition comprising said immune cells of the disclosure is administered in a prophylactically effective amount.

The immune cells or pharmaceutical compositions of the disclosure may be administered in a number of ways depending upon whether local or systemic treatment is desired. The NK cells and macrophages, or pharmaceutical compositions thereof, are typically suitable for parenteral administration, wherein administration includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intranasal, intratracheal, intraarterial, intrathecal, intraventricular, intraurethral, intracranial, intratumoral, intraocular, intradermal, intrasynovial injection or infusions, intra-tumoral; and kidney dialytic infusion techniques. In some embodiments, the immune cells, or pharmaceutical compositions of the present disclosure comprise intravenous administration. In some embodiments, the immune cells, or pharmaceutical compositions of the present disclosure comprise intra-tumoral administration. In some embodiments, the immune cells, or pharmaceutical compositions are administered to a patient in a similar fashion to previous clinical work with immune cell-based therapies using unmodified peripheral blood immune, or NK and macrophage, cells.

In some embodiments, the engineered immune cell or pharmaceutical composition comprising said immune cells of the disclosure are administered in combination with a combination partner. The term “combination” refers to either a fixed combination in one dosage unit form, or a kit of parts for the combined administration where the immune cells, or pharmaceutical composition comprising the combination of said NK and macrophage cells of the disclosure, and a combination partner (e.g., an antibody or another drug as explained below, also referred to as “therapeutic agent” or “co-agent”) may be administered independently at the same time or separately within time intervals. In some circumstances the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

In some embodiments, the administering further comprises administering the immune cells or pharmaceutical composition comprising said engineered immune cell in combination with an antibody specific to a disease. In some embodiments, the antibody specific to a disease is an anti-CD47, anti-PD1, anti-PDL1 or anti-CTLA4 antibody. In some embodiments, the anti-CD20 antibody is rituximab. In some embodiments, the antibody specific to a disease is an anti-EGFR antibody. In some embodiments, the anti-EGFR antibody is cetuximab. Such combination may lead to antibody dependent cell cytotoxicity (ADCC) and or antibody dependent cell phagocytosis (ADCP).

As used herein, “antibody” is understood to mean any antigen-binding molecule or molecular complex comprising at least one complementarity determining region (CDR) that binds specifically to, or interacts specifically with, the target antigen. The term “antibody” includes full-length immunoglobulin molecules comprising two heavy (H) chains and two light (L) chains inter-connected by disulfide bonds, as well as multimers thereof (e.g., IgM). Each heavy chain comprises a heavy chain variable region (which may be abbreviated as HCVR, VH or VH) and a heavy chain constant region. The heavy chain constant region typically comprises three domains—CH1, CH2 and CH3. Each light chain comprises a light chain variable region (which may be abbreviated as LCVR, VL, VK, VK or VL) and a light chain constant region. The light chain constant region will typically comprise one domain (CL1). The VH and VL regions can be further subdivided into regions ofhypervariability, termed complementarity determining regions (CDRs), interspersed with regions that are more conserved, also referred to as framework regions (FR).

As described herein, “antigen-binding molecule” is an antibody or an antigen binding fragment thereof, as described elsewhere herein. In an embodiment, the antigen binding fragment is selected from the group consisting of a Fab fragment, scFab, Fab′, a single chain variable fragment (scFv) and a one-armed antibody.

Non-limiting examples of suitable antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated CDR such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g. monovalent nanobodies, bivalent nanobodies, etc.), and small modular immunopharmaceuticals (SMIPs), are also encompassed by the term “antigen-binding fragment,” as used herein.

As used herein, the term “complementarity determining region” (CDR) refers to the region of an immunoglobulin variable domain that recognizes and binds to the target antigen. Each variable domain may comprise up to three CDR sequences, identified as CDR1, CDR2 and CDR3.

The phrase “specifically binds” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antigen-binding molecule binds to a particular antigenic determinant, thereby identifying its presence. Specific binding to an antigenic determinant under such conditions requires an antigen-binding molecule that is selected for its specificity to that determinant. This selection may be achieved by subtracting out antigen-binding molecules that cross-react with other molecules. A variety of immunoassay formats may be used to select antigen-binding molecules (e.g., immunoglobulins) [such that they are specifically immunoreactive with a particular antigen]. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are also well known in the art (see, for example, Harlow and Lane, supra); Friefelder, “Physical Biochemistry: Applications to biochemistry and molecular biology” (W.H. Freeman and Co. 1976).

Antibodies may include, but are not limited to, monoclonal antibodies (including full length monoclonal antibodies), polyclonal antibodies, multi-specific antibodies (e.g., bi-specific antibodies), and antibody fragments so long as they exhibit the desired biological activity of binding to a target antigenic site and its isoforms of interest. The term “antibody fragments” comprise a portion of a full length antibody, generally the antigen binding or variable region thereof. The term “antibody” as used herein encompasses any antibodies derived from any species and resources, including but not limited to, human antibody, rat antibody, mouse antibody, rabbit antibody, and so on, and can be synthetically made or naturally-occurring.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the art.

As used herein, the term “isolated” is used to refer to molecules or cells that are removed from native environments. As used herein, the term “non-naturally occurring” is used to refer to isolated molecules or cells that possess markedly different structures than counterparts found in nature.

The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. As used herein, a “chimeric protein” or “fusion protein” comprises a first polypeptide operatively linked to a second polypeptide. Chimeric proteins may optionally comprise a third, fourth or fifth or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art.

In some embodiments, the subject in need thereof has or is believed to have a malignancy. Many types of malignancies can develop resistance mechanisms to evade attacks from endogenous NK cells, nonlimiting examples are provided herein. In some embodiments, the malignancy may include Acute Lymphoblastic Leukemia (ALL), Acute Myeloid Leukemia (AML), Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Astrocytomas, Atypical Teratoid/Rhabdoid Tumor, Basal Cell Carcinoma of the Skin, Bile Duct Cancer, Bladder Cancer, Bone Cancer (includes Ewing Sarcoma and Osteosarcoma and Malignant Fibrous Histiocytoma), Brain Tumors, Breast Cancer, Bronchial Tumors, Burkitt Lymphoma, Carcinoid Tumor, Carcinoma, Cardiac Tumors, Atypical Teratoid/Rhabdoid Tumor, Medulloblastoma, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ (DCIS), Endometrial Cancer, Ependymoma, Esophageal Cancer, Esthesioneuroblastoma, Ewing Sarcoma, Extracranial Germ Cell Tumor, Extragonadal Germ Cell Tumor, Intraocular Melanoma, Retinoblastoma, Fallopian Tube Cancer, Fibrous Histiocytoma of Bone, Osteosarcoma, Gallbladder Cancer, Gastric Cancer, Gastrointestinal Carcinoid Tumor, Gastrointestinal Stromal Tumors (GIST) (Soft Tissue Sarcoma), Germ Cell Tumors, Central Nervous System Germ Cell Tumors, Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Hepatocellular Cancer, Histiocytosis (Langerhans Cell), Hodgkin Lymphoma, Hypopharyngeal Cancer, Islet Cell Tumors, Pancreatic Neuroendocrine Tumors, Kaposi Sarcoma (Soft Tissue Sarcoma), Renal Cell Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and Tracheobronchial Tumor), Lymphoma, Male Breast Cancer, Malignant Fibrous Histiocytoma of Bone and Osteosarcoma, Merkel Cell Carcinoma, Mesothelioma, Metastatic Cancer, Metastatic Squamous Neck Cancer with Occult Primary, Midline Tract Carcinoma With NUT Gene Changes, Oropharyngeal Cancer, Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Chronic Myelogenous Leukemia (CML), Myeloid Leukemia, Acute (AML), Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Pancreatic Cancer, Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal Cancer, Prostate Cancer, Recurrent Cancer, Rhabdomyosarcoma, Salivary Gland Cancer, Vascular Tumors, Small Intestine Cancer, Soft Tissue Sarcoma, T-Cell Lymphoma, Thymoma and Thymic Carcinoma, Transitional Cell Cancer of the Renal Pelvis and Ureter, Vaginal Cancer, Vulvar Cancer, or Wilms Tumor.

In some embodiments, the malignancy may comprise tumor-associated antigens. In some embodiments, the malignancy may comprise a cell marker characteristic of a malignancy. In some embodiments, the cell marker characteristic of a malignancy is a tumor-associated antigen, receptor, or other protein or structure attributed to cells with cancerous phenotypes.

Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGEA1, MAGE-A2, MAGE-A3, MAGE-A4, MAGE-A6, MAGE-A10, MAGE-A12, MART-1, BAGE, DAM-6, DAM-10, GAGE-1, GAGE-2, GAGE-8, GAGE-3, GAGE-4, GAGE-5, GAGE-6, GAGE-7B, NA88-A, MART-1, MC1R, GplOO, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, β-catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (e.g., such as EGFRvIII), platelet derived growth factor receptor (PDGFR), vascular endothelial growth factor receptor (VEGFR), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin Bl, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1, melanocyte melanoma lineage antigens (e.g., MART-1/Melan-A, gp75, mda-7, tyrosinase and tyrosinase-related protein), HER-2/neu, and idiotypes.

In some embodiments, the malignancy, or cells thereto, exhibit CD19, CD20, Her2, CD19, CD319/CS1, ROR1, CD20, CD5, CD7, CD22, CD70, CD30, BCMA, CD25, NKG2D ligands, MICA/MICB, carcinoembryonic antigen, alphafetoprotein, CA-125, MUC-1, epithelial tumor antigen, melanoma-associated antigen, mutated p53, mutated ras, HER2/Neu, ERBB2, folate binding protein, HIV-1 envelope glycoprotein gpl20, HIV-1 envelope glycoprotein gpl41, GD2, CD123, CD33, CD30, CD56, c-Met, mesothelin, GD3, HERV-K, IL-llRalpha, kappa chain, lambda chain, CSPG4, ERBB2, WT-1, EGFRvIII, TRAIL/DR4, VEGFR2, PTK-7, B7H3, PD-L1, CD38, CLL-1, LeY, CAIX, CD133, CD171, GPC3, CEA, Ep-CAM, EphA2, FAP, HPV16-E6, ILI3Ra2, MAGEA3, MAGEA4, MART1, MUC16, NY-ESO-1 and/or PSCA, CLL-1/CLECi2A, BCMA, TROP2, Nectin-4, CD79b, CD2, CD3, CD4, PD-1, KIR2DL3, ALPPL2, or CSP1.

In some embodiments, the subject in need thereof has or is believed to have a viral infection. In some embodiments, the viral infections mammalian viral infection. Examples of mammalian viral infections include, but are not limited to: infections caused by DNA Viruses (e.g., Herpes Viruses such as Herpes Simplex viruses, Epstein-Barr virus, Cytomegalovirus; Pox viruses such as Variola (small pox) virus; Hepadnaviruses (e.g, Hepatitis B virus); Papilloma viruses; Adenoviruses); RNA Viruses (e.g., HIV I, II; HTLV I, II; Poliovirus; Hepatitis A; Orthomyxoviruses (e.g., Influenza viruses); Paramyxoviruses (e.g., Measles virus); Rabies virus; Hepatitis C); Coronavirus (causes Severe Acute Respiratory Syndrome (SARS)); Rhinovirus, Respiratory Syncytial Virus, Norovirus, West Nile Virus, Yellow Fever, Rift Vallley Virus, Lassa Fever Virus, Ebola Virus, and Lymphocytic Choriomeningitis Virus. In some embodiments, the viral infection is acute. In some embodiments, the viral infection is chronic.

Cells infected with a virus may present with viral infection-associated antigens. Nonlimiting examples of viral infection-associated antigens include, but are not limited to, core protein (C protein), non-structural protein 3 (NS3), non-structural protein 5 (NS5), enveloped protein (E protein), non-structural protein 4 (NS4), hemagglutinin (HA), nucleoprotein (NP), neuraminidase (NA), matrix protein 1 (M1), F protein, N protein, G protein, capsid protein (C), non-structural protein (NS), envelop protein (E), precursor membrane protein (prM), non-structural protein 1 (NS1), Gag, Env, Tat, Pol, Nef, Vif, capsid protein P1 (VP2), capsid protein P1 (VP1), and capsid protein P1 (VP3).

In an aspect, the disclosure provides a cellular culture comprising Natural Killer (NK) cells and macrophages. In embodiments, the NK cells and/or macrophages have been produced from induced pluripotent stem cells (iPSCs). In embodiments, the NK cells and/or macrophages have been produced from peripheral blood cells or cord blood cells. In embodiments, the cells are human cells.

In some embodiments, the NK cells exhibit enhanced antibody-dependent cellular cytotoxicity (ADCC) as compared to wildtype NK cells, and particularly in combination with macrophages.

In an aspect, the disclosure provides a pharmaceutical composition comprising NK cells and/or macrophages from the culture of cells as described herein.

In an aspect, the disclosure provides a method of treating a subject in need comprising administering to the subject an effective amount of a pharmaceutical composition as described herein. In embodiments, the invention provides that the subject in need has a NK-resistant cancer. In embodiments, the invention provides that the subject in need has a chronic viral infection.

In embodiments, the administration further includes antibodies specific for a diseased cell. In embodiments, the invention provides that the administration further includes antibodies specific for an immune regulatory pathway, including anti-CD47, anti-PD1, anti-PDL1 or anti-CTLA40 antibodies.

The disclosure is now described with reference to the following Examples. These Examples are provided for the purpose of illustration only and the disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the methods of the present disclosure and practice the claimed methods. The following working examples therefore, specifically point out embodiments of the present disclosure, and are not to be construed as limiting in any way the remainder of the disclosure.

### Examples

The materials and methods employed in these experiments are now described.

Despite many advances, the treatment of acute myeloid leukemia (AML) remains challenging, and few patients are cured by therapies other than allogeneic hematopoietic cell transplant. Treatment with natural killer (NK) cells from allogeneic donors is a promising therapy that can achieve remissions in 30-50% of AML patients. To generate an improved cell based therapy for AML, NK cells have been produced from induced pluripotent stem cells (iPSCs). iPSC-derived NK cells effectively kill AML cells, but may benefit from additional modifications or combination with other therapies to durably cure AML. Based on studies that demonstrate that targeting the CD47 pathway on macrophages and NK cells improves anti-tumor activity and is an effective treatment for patients with AML, the combination of iPSC-derived NK cells with iPSC-derived macrophages with and without CD47 blockade for the treatment of AML may be a promising method of treating AML.

AML clinical trials combining anti-CD47 monoclonal antibodies (mAb) with chemotherapy have demonstrated an antitumor effect primarily thought to be mediated though a macrophage immune-checkpoint blockade mechanism. To determine if the addition of iPSC-derived macrophages can improve the cytotoxicity of NK cells against AML blasts, iPSC-NK cells and MOLM13 or MV-4-11 AML cells were co-cultured with iPSC-macrophages in a standard cytotoxicity assay. Similar results were found for cytotoxicity tests against both AML cell lines. While macrophages alone did not kill AML blasts, the addition of iPSC-macrophages to iPSC-NK cells significantly improved killing of AML blasts by 50% (p<0.01). Addition of an anti-CD47 mAb (B6H12) further increased killing of AML blasts by the iPSC-NK cell+iPSC-macrophage combination treatment by an additional 23% (p<0.01). Intriguingly, the addition of just the anti-CD47 mAb to the iPSC-NK cells also significantly increased killing of AML cells, although this increased killing was consistently lower than what was seen with the addition of iPSC-macrophages combined with anti-CD47 and iPSC-NK cells. Addition of the CD47 mAb to iPSC-macrophages without NK cells did not result in increased anti-AML activity. It was also demonstrated that blockade of SIRPα (the receptor for CD47 on NK cells) significantly increased NK cell killing of AML blasts by 16% (p<0.05). Furthermore, the combination of iPSC-NK cells+iPSC-macrophages+SIRPα mAb led to a 37% increase in cytotoxicity compared to iPSC-NK cells+iPSC-macrophages alone (p<0.01).

To confirm that addition of anti-CD47 or anti-SIRPα antibodies increased NK cell activation via loss of the inhibitory CD47-SIRPα interaction between NK cells and AML blasts and not via another mechanism, the effect of adding CD47 mAb and anti-SIRPα antibodies simultaneously was tested. Compared to addition of either anti-CD47 or anti-SIRPα mAb alone, the combination induced no additional increase in anti-AML activity by the NK cells. These results suggest that the CD47-SIRPα interaction between AML and NK cells is an important inhibitory immune-checkpoint on NK cells. To control for the effect of blocking SIRPα on macrophages, the addition of the SIRPα mAb to iPSC-macrophages was tested. This combination did not improve on the lack of cytotoxicity exhibited by macrophages alone. Whether ADCC mediated by the anti-CD47 mAb binding AML blasts could account for the increase in cytotoxicity was also evaluated, and it was found that blockade of Fc-receptors on NK cells does not diminish the increase in cytotoxicity seen with addition of the mAb, excluding a role for ADCC.

To investigate if these findings with AML could be extended to other hematologic malignancies, the combination of iPSC-NK cells, iPSC-macrophages and CD47 mAb against the RPMI-8226 multiple myeloma cell line was tested. Here, it was again demonstrated that CD47 blockade combined with iPSC-macrophages leads to increased NK cell-mediated anti-myeloma activity. In vivo studies testing the combination of iPSC-NK cells, iPSC-macrophages and CD47 mAb against human AML and myeloma cells in mouse xenograft-models are ongoing. Together these results indicate that blocking the CD47-SIRPα interaction between NK cells and tumor cells consistently mediates improved anti-tumor activity. Furthermore, iPSC derived NK cells and macrophages provide an important, standardized, “off-the-shelf” cell therapy approach that can be translated into novel clinical therapies.

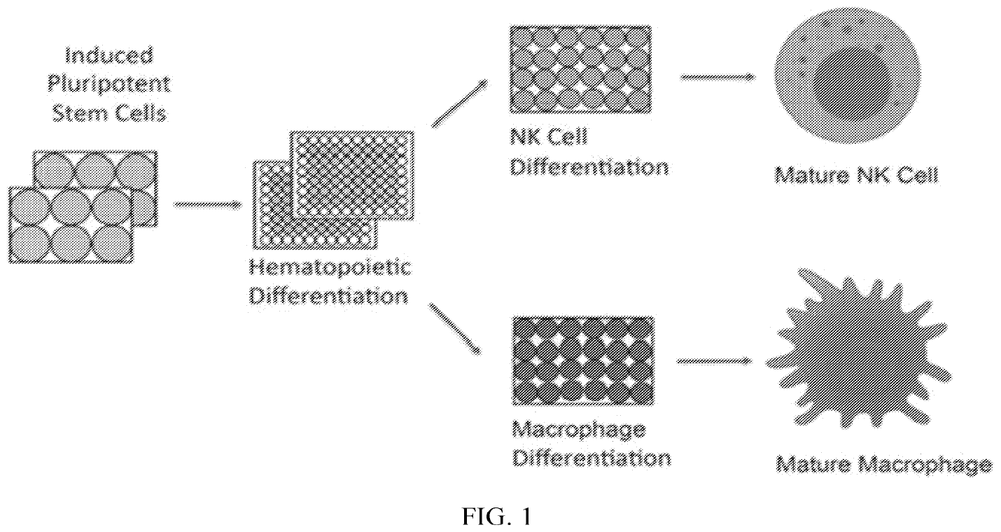

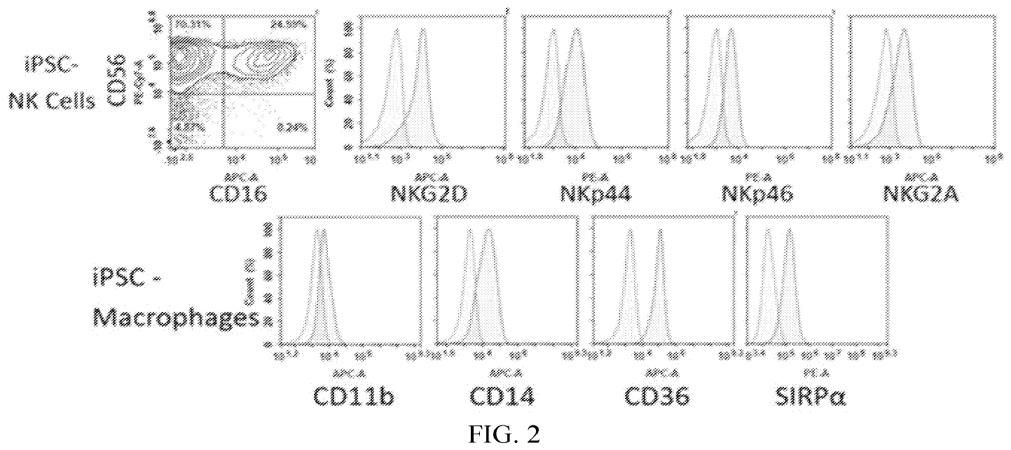

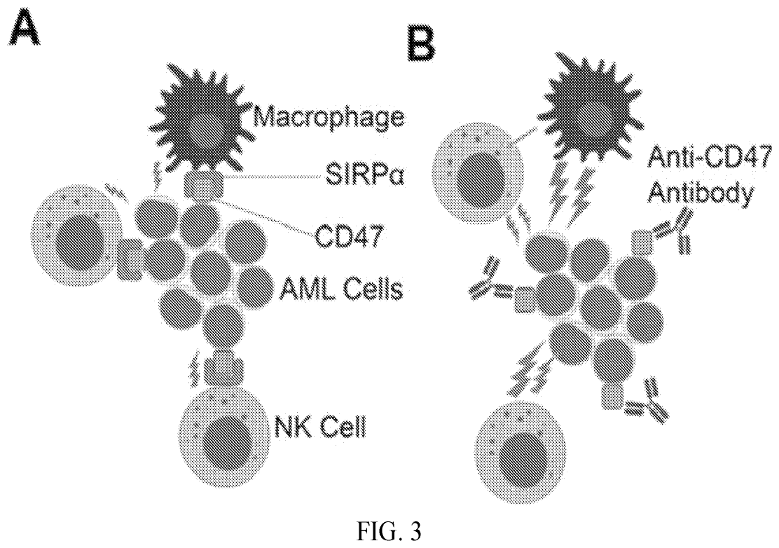

In embodiments, as shown in FIGS. 1-2, human iPSCs serve as a renewable source of iPSC-derived NK cells and macrophages that can be easily scaled up for therapeutic applications. The data demonstrate that administration of human iPSC-derived NK cells and macrophages together, along with therapeutic antibodies blocking the CD47-SIRP interaction increases killing of AML and multiple myeloma cells, as shown in FIGS. 3-5. Other immune regulatory agents such as check point inhibitors that block PD1, PDL1 or CTLA4 can also be used. The combination of NK cells and macrophages increase killing compared to NK cells alone and efficacy of the combination cell therapy treatment is further enhanced by addition of antibodies blocking the CD47-SIRP interaction.

Adoptive cell therapy treatments for hematologic malignancies such as B-cell leukemias and lymphomas and multiple myeloma have demonstrated strong efficacy with five chimeric antigen receptor (CAR) T cell products now approved by the FDA. Alternative effector cells such as natural killer (NK) cells and macrophages have also been heavily studied in pre-clinical and clinical trials. Adoptive transfer of NK cells and macrophages have multiple advantages over T cells including that they function as allogeneic immune cells, and do not require derivation or isolation on a patient specific basis. NK cells are an ideal cell population for anti-cancer cell therapy as they are activated by, recognize and kill tumor cells without the requirement for antigen specific sensitization and allogeneic NK cells do not cause graft-versus-host disease. Macrophages are one of the most important phagocytic cells in the human immune system and macrophages also serve as antigen presenting cells and secrete cytokines that stimulate endogenous NK cell and T cell activity.

Importantly, protocols have been developed to differentiate NK cells and macrophages from human pluripotent stem cells including from induced pluripotent stem cells (iPSCs) or human embryonic stem cells (hESCs), as shown in FIG. 1. Specifically, iPSCs are first dissociated, resuspended in hematopoietic differentiation media, and centrifuged to form spin embryoid bodies that differentiate into hematopoietic progenitor cells. The hematopoietic progenitor cells can then be differentiated into either a mature NK cell or a macrophage fate depending on the media and cytokine conditions used.

Pluripotent stem cells, particularly iPSCs, serve as an excellent platform for cellular genetic engineering to enhance anti-tumor activity. The invention provides genetically engineered iPSC-derived NK cells and macrophages expressing CARs that demonstrate improved targeted killing of both hematologic malignancies and solid tumors in vitro and in vivo, as shown in FIG. 2. To assess the efficacy of targeted killing of tumor cells, iPSC-NK cells and iPSC-macrophages were stained and analyzed by flow cytometry for the indicated NK cell and macrophage cell surface receptors. Other genetic modification such as knockout of negative regulators of signaling pathways in NK cells also have shown increased anti-tumor activity.

There are two important aspects to consider with respect to the improved anti-tumor efficacy seen with the combination treatment described herein. The first is the interaction between NK cells and macrophages that leads to increased cytotoxicity against tumors. The second is blockade of the CD47-SIRP interaction on NK cells and macrophages with a monoclonal antibody that also increases tumor killing.

The combination treatment with NK cells and macrophages that leads to increased cytotoxicity against tumors may work through physical interactions between the two cell types or may be mediated by soluble factors such as cytokines produced from either (or both) cell populations. The data show little direct killing of tumor cells by macrophages, making it more likely that macrophages are enhancing NK cell tumor killing. Macrophages are known to present activating ligands directly to NK cells and to release cytokines that stimulate NK cell activity. Both mechanisms are likely to play a role in the enhanced killing disclosed herein.

Blockade of the CD47-SIRP interaction on NK cells and macrophages likely works by decreasing inhibitory signaling through SIRP, as shown in FIGS. 3A-3B. FIG. 3A demonstrates that the combination therapy of iPSC-NK cells and iPSC macrophages are able to kill AML cells than either cell population alone. In doing so, CD47 on the AML cells bind to SIRPα on NK cells and macrophages and provides an inhibitory signaling that inhibits immune cell activation and leukemia killing. Similarly, as shown in FIG. 3B, blockade of the CD47-SIRPα interaction by the anti-CD47 antibody removes the inhibitory signaling mediated by SIRPα on NK cells and macrophages and increases killing of AML cells.

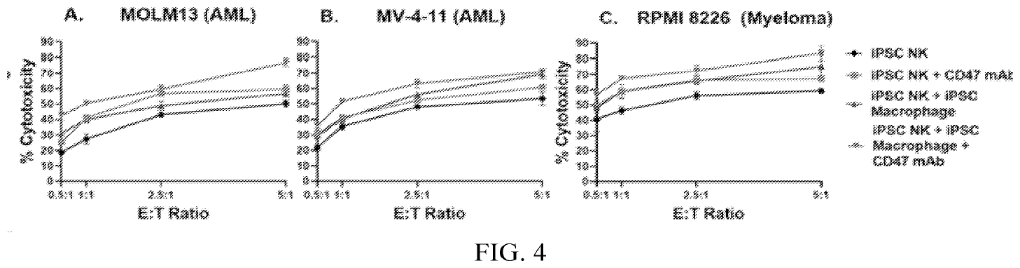

NK cells and macrophages both express SIRP receptors that produce inhibitory signaling when bound to CD47 on tumor cells. Blocking this inhibitory signaling pathway increases tumor killing of NK cells and activation of macrophages, as shown in FIGS. 4A-4C. For example, FIGS. 4A-4B show that the combination of iPSC-NK cells and iPSC-macrophages is effective to increase killing of AML cells, whereas FIG. 4C shows that this combination is also effective to increase killing of multiple myeloma cells. Notably, addition of an anti-CD47 mAb (B6H12) further increased the killing of AML blasts by the combination treatment of iPSC-NK cells and iPSC-macrophages.

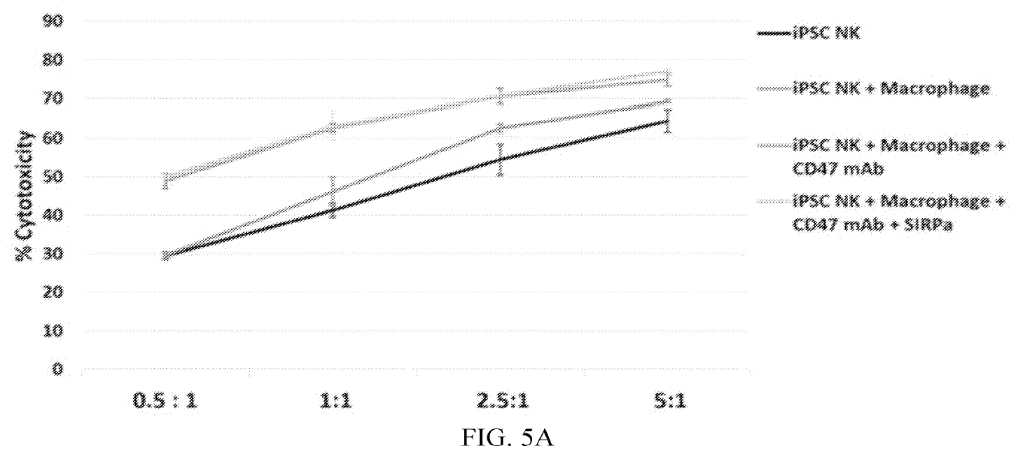

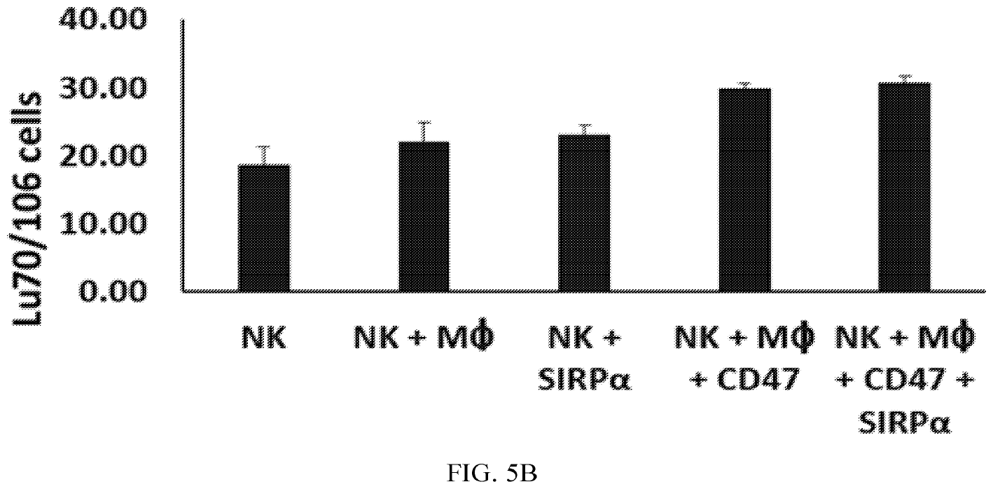

This idea is further supported by the fact that blocking antibodies to the SIRP receptor produces similar results as the CD47 blocking antibody, as shown in FIGS. 5A-5B. The combination of iPSC-NK cells and iPSC-macrophages increased killing of AML cells compared to NK cells alone. Specifically, addition of an anti-CD47 mAb increased killing of AML blasts, but there was no further increase in killing with addition of the anti-SIRPα antibody to the anti-CD47 antibody. This supports the non-limiting hypothesis that the anti-SIRPα and anti-CD47 antibodies act through the same pathway.

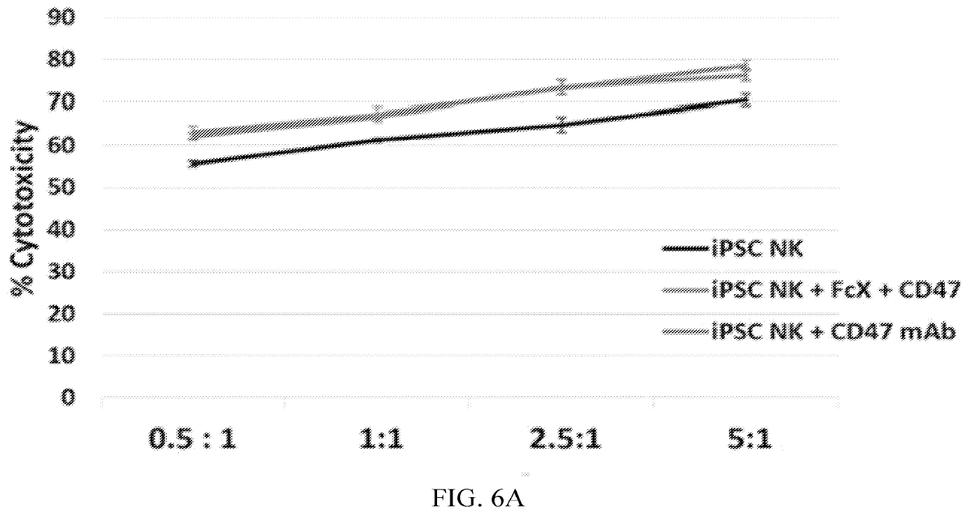

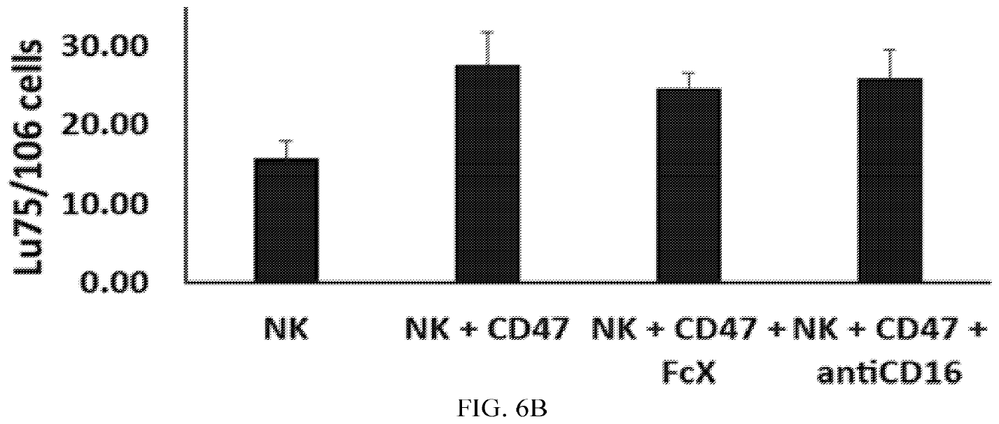

A second possible non-limiting mechanism for the activity of the anti-CD47 antibody is that it directly increases killing to tumor cells it binds via antibody dependent cellular cytotoxicity. Human iPSC-derived NK cells and macrophages express Fc receptors necessary to mediate antibody dependent cellular cytotoxicity (ADCC). Administration of the anti-CD47 antibody here could cross-link the FcRs more efficiently and trigger signals which lead to ADCC of tumor cells. This is less likely due to the fact that blocking of Fc receptors does not significantly change the increased killing seen with administration of the CD47 antibody, as shown in FIG. 6A-6B. Blocking of Fc receptors did not modify the increased killing seen with administration of the anti-CD47 antibody. NK cells were treated with Fc receptor blocking reagents prior to co-incubation with AML cells that were treated with the anti-CD47 blocking antibody.

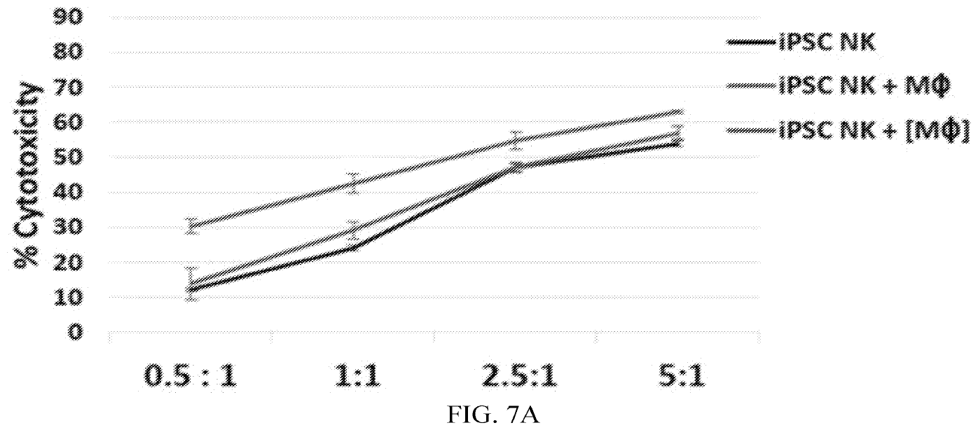

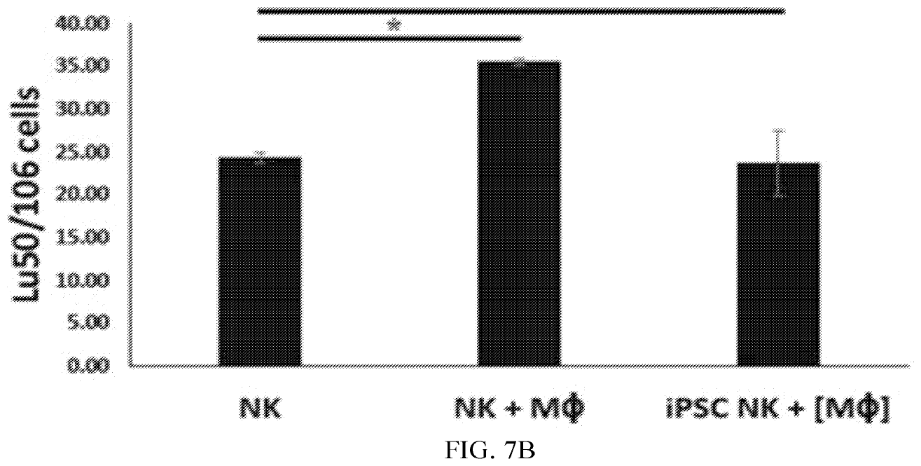

Moreover, it was observed that for optimal efficacy of the combination therapy of iPSC-NK cells and iPSC-macrophages arises when there is physical contact between the immune cell populations, as shown in FIGS. 7A-7B. iPSC-NK cells were co-incubated with AML blasts either with iPSC-macrophages together or with iPSC-macrophages separated by a transwell insert (indicated by [Mφ]). The increase in cytotoxicity mediated by iPSC-NK cells seen with the addition of iPSC-macrophages was significantly reduced when the iPSC-macrophages were separated by the transwell insert.

What is claimed is:

1. A method of treating a disease or disorder in a subject in need thereof, comprising administering to the subject an effective amount of natural killer (NK) cells and macrophages.

2. The method of claim 1, wherein the NK cells and the macrophages are co-incubated prior to being administered, thereby improving the efficacy of the NK cells and the macrophages in treating the disease or disorder in the subject.

3. The method of claim 1, further comprising administering to the subject an effective amount of an antibody blocking an immune regulatory pathway.

4. The method of claim 3, wherein the antibody is anti-CD47, anti-PD1, anti-PDL1 or anti-CTLA4.

5. The method of claim 1, wherein the subject suffers from a malignancy.

6. The method of claim 5, wherein the malignancy is AML and/or multiple myeloma.

7. The method of claim 1, wherein the NK cells and the macrophages are derived from induced pluripotent stem cell (iPSC)-derived immune cells.

8. The method of claim 1, wherein the NK cells and the macrophages are derived from peripheral blood (PB)-derived immune cells.

9. The method of claim 1, wherein the NK cells and the macrophages are derived from cord blood (CB)-derived immune cells.

10. The method of claim 1, wherein the NK cell or the macrophages are engineered.

11. A purified cell composition comprising at least partially isolated natural killer (NK) cells and macrophages.

12. The purified cell composition of claim 11, further comprising an antibody blocking an immune regulatory pathway.

13. The purified cell composition of claim 11, wherein the antibody is anti-CD47, anti-PD1, anti-PDL1 or anti-CTLA4.

14. The purified cell composition of claim 11, wherein the purified cell composition is effective to treat a malignancy in a subject.

15. The purified cell composition of claim 14, wherein the malignancy is AML and/or multiple myeloma.

16. A pharmaceutical composition comprising an effective amount of natural killer (NK) cells and macrophages.

17. The pharmaceutical composition of claim 16, further comprising an effective amount of an antibody blocking an immune regulatory pathway.

18. The pharmaceutical composition of claim 17, wherein the antibody is anti-CD47.

19. The pharmaceutical composition of claim 16, wherein the NK cells and macrophages are effective to treat a malignancy in a subject.

20. The pharmaceutical composition of claim 16, wherein the NK cells and the macrophages are derived from induced pluripotent stem cell (iPSC)-derived immune cells.
